(19) United States
(12) Patent Application Publication
(10) Pub. No.: US 2023/0030355 A1
(43) Pub. Date: Feb. 2, 2023

(54) CRYOTHERAPY DEVICE

(71) Applicant: CET LIMITED, Loughborough (GB)

(72) Inventor: Robert Colin Edgar, Dromore, County Down (GB)

(73) Assignee: CET LIMITED, Loughborough (GB)

(21) Appl. No.: 17/758,454

(22) PCT Filed: Nov. 9, 2020
(86) PCT No.: PCT/EP2020/081433

§ 371 Date: Jul. 6, 2022

(30) Foreign Application Priority Data
Jan. 8, 2020 (GB) 2000224.2

(51) Int. Cl. A61F 7/00; A61H 9/00

(52) U.S. Cl. CPC A61F 7/0053; A61F 7/0085; A61H 9/0021; A61F 2007/0036; A61F 2007/0045; A61F 2007/0063; A61F 2007/0093; A61H 2033/021

(57) ABSTRACT

The present invention relates to a device for manipulating core body temperature of a human subject by convection and conduction, the device comprising: an open topped liquid carrier vessel adapted to receive a liquid, the liquid carrier vessel having an inlet and an outlet in the base thereof; a removable splash guard for covering the majority of the liquid carrier vessel opening and for resting thereon of a human extremity having an exposed glabrous skin surface; chiller means for cooling liquid received in the vessel and for maintaining the liquid at a predetermined temperature; a jet in the vessel inlet for discharging liquid, or a combination of liquid and air into the liquid carrier vessel; an air valve for controlling the amount of air flowing through the jet; a pump configured to suck liquid from the liquid carrier vessel via the vessel outlet into a suction side of the pump and propel liquid out of a pressure side of the pump into the liquid carrier vessel through the vessel inlet via the chiller means to create a jet stream within the liquid carrier vessel at the predetermined temperature; first conduit means interconnecting the vessel outlet with the suction side of the pump; second conduit means interconnecting the chiller means with the pressure side of the pump; third conduit means interconnecting the chiller means with the jet; and fourth conduit means interconnecting the jet with the air valve; wherein the splash guard is provided with an aperture to allow the jet stream to contact the exposed glabrous skin surface of the subject during operation of the device; and wherein when the splash guard is in place, the aperture is located directly above the jet.

The present invention relates to the field of sports recovery, in particular to a device for manipulating core body temperature.

### BACKGROUND TO THE INVENTION

Sports injuries have been treated with ice for many years. Ice baths and ice wraps have been shown to be particularly effective in relieving muscle pain, sprains and swelling after soft tissue damage. More recently, low temperature cryotherapy spas have been used for sports recovery and rehabilitation as an alternative to ice baths. These cryotherapy spas involve immersion of an athlete's body, typically to waist depth, or injured body part or parts in chilled, whirling water. Water jets are typically used to create the whirling water movement.

Whilst these prior art methods are very good for both recovery after exercise and for the treatment of soft tissue injuries, they cannot be used to enhance the athlete's ability to perform physical exercise during a short break in exercise as they are not usually portable and typically involve removal of the injured sports person's clothing. Furthermore, as such methods involve the cooling of major muscle groups, risk of injury to a subject exercising immediately after treatment is high due to exertion while tissue is still cold. In other words, they cannot be used during in-match intervals.

One type of device which sets out to achieve portable sports recovery while not cooling major muscle groups is the vacuum glove disclosed in European Patent No. EP 1278490. This device works by enclosing an athlete's limb, for example the lower arm, and contacting the surface of the enclosed portion with a low temperature medium under negative pressure conditions such that the skin is not in direct contact with the cooling medium.

One disadvantage with this device and others which use negative pressure is that is the cooling is relatively slow and in situations where time is limited this is a distinct disadvantage. Another disadvantage is that the skin of the subject using the device is only cooled by conduction, which significantly limits the cooling effect. Furthermore, the noise output from the above prior art device in use is high.

It is a therefore an object of the present invention to provide an improved device for manipulating core body temperature and a suitable alternative.

### SUMMARY OF THE INVENTION

The present invention relates to a device for manipulating an individual's core body temperature using both convection and conduction in order to speed up the individual's recovery after strenuous exercise, i.e. exercise which has resulted in raised core body temperature, while not compromising sporting performance directly after use of the device. For example, the device may be used during half-time in a hockey match, rugby match, football match or the like.

Accordingly, a first aspect of the present invention is a device for manipulating core body temperature of a human subject by convection and conduction, the device comprising:
an open topped liquid carrier vessel adapted to receive a liquid, the liquid carrier vessel having an inlet and an outlet in the base thereof;
a removable splash guard for covering the majority of the liquid carrier vessel opening and for resting thereon of a human extremity having an exposed glabrous skin surface;
chiller means for cooling liquid received in the vessel and for maintaining the liquid at a predetermined temperature;
a jet in the vessel inlet for discharging liquid or a combination of liquid and air into the liquid carrier vessel;
an air valve for controlling the amount of air flowing through the jet;
a pump configured to suck liquid from the liquid carrier vessel via the vessel outlet into a suction side of the pump and propel liquid out of a pressure side of the pump into the liquid carrier vessel through the vessel inlet via the chiller means to create a jet stream within the liquid carrier vessel at the predetermined temperature;
first conduit means interconnecting the vessel outlet with the suction side of the pump;
second conduit means interconnecting the chiller means with the pressure side of the pump;
third conduit means interconnecting the chiller means with the jet; and
fourth conduit means interconnecting the jet with the air valve;

wherein the splash guard is provided with an aperture to allow the jet stream to contact the exposed glabrous skin surface of the subject during operation of the device; and

wherein when the splash guard is in place, the aperture is located above the jet.

The device rapidly impacts the core temperature of a subject's body using a high thermal conductivity medium without over-stimulating the body's automatic reflex mechanisms such as vasoconstriction in a cold environment, e.g. with an ambient temperature of less than 15° C. This avoidance of vasoconstriction is achieved by cooling the palm of the hand or other glabrous (non-hairy) part or parts of the body, by both conduction and convection using a medium with a high thermal conductivity—the thermal conductivity of water, for example, being 26 times greater than air—combined with vigorous massage. This provides significant advantages over prior art.

In order to use the device according to the invention, a subject place a glabrous skin surface over the aperture in the splash guard. Upon operation of the device, a jet stream is generated which, due to the fact that the aperture is located above the jet, contacts the exposed glabrous skin surface and creates a massaging effect.

Without being bound by theory, use of the device according to the invention triggers transient receptor potential channels (TRPs) in the skin, which are connected via the spinal column to the brain, to give the subject a mood elevating boost and prepare them mentally as well as physically for the next challenge. TRPs are triggered by temperature, pressure, vibration and certain chemicals and stimulate the production of mood elevating chemicals in the brain such as serotonin and dopamine.

The subject is preferably a human, particularly preferably an elite athlete such as a professional football player. However, this is not considered to be limiting and the device may be used for impacting the core body temperature of any subject having a glabrous skin surface. For example, the device may be used by firefighters and military personnel wearing chemical protection suits when their activities may cause their core body temperature to reach dangerous levels. Alternatively, the device may be used to moderate an individual's core body temperature where, due to illness or traumatic stress, their core body temperature has been raised or lowered to such a degree as to compromise normal physiological performance, for example for treatment of certain conditions such as multiple sclerosis where the patient has difficulty regulating their core body temperature.

By glabrous surface is meant a skin surface that is characterised by an absence of hair follicles and the presence of unique subcutaneous vascular structures called venous plexuses that enable a large volume of the circulating blood to flow directly below the skin surface via arterio-venous anastomoses (AVAs) that provide a means of thermoregulatory control. Glabrous skin surfaces to which the invention is to be applied preferably include the palm of the hand and the sole of the foot.

The liquid carrier vessel is open at the top and is in the form of a shell with a well, for example like a small bathtub or basin, e.g. an elongated trough. The liquid carrier vessel preferably includes one or more liquid level indicators indicating minimum and maximum liquid fill levels for the device. Each liquid level indicator independently may, for example, be an indentation or raised area of a sidewall of the vessel. However, this is not to be considered limiting and other suitable liquid level indicators may be used.

The liquid carrier vessel of the device is preferably made of plastic, glass fibre, carbon fibre or, less preferably metal, or a combination thereof. Plastic or glass fibre lends itself best to this type of product.

The internal dimensions of the liquid carrier vessel are preferably in the range of from about 30 to about 45 cm long, about 25 to about 40 cm wide and about 15 to about 30 cm deep. However, these ranges are not to be considered limiting and the vessel may be larger or smaller depending on the intended number of simultaneous users.

Ideally, the volume capacity of the liquid carrier vessel is in the range of from about 5 to about 20 litres. However, this is not to be considered limiting and the volume capacity may be larger or smaller depending on the intended number of simultaneous users.

The liquid vessel carrier may have straight or curved walls and may include a stepped portion such that a top portion is larger than a bottom portion, forming a ledge on the internal surface of the vessel. The ledge may be continuous or discontinuous. For example, the vessel may have two straight walls and two stepped walls such that the stepped walls oppose each other. In the embodiments where the vessel has a stepped portion, the splash guard is placed on the ledge such that the top portion of the liquid carrier vessel extends above the splash guard.

The splash guard gives the subject something to rest, for example their hand or arm, on during use to increase comfort. Also, the splash guard, as the name suggests, guards against the inadvertent splashing of liquid on the subject generally.

The splash guard is preferably made of glass or solid transparent plastic, for example Plexiglas or Perspex. However, this is not to be considered limiting. The splash guard does not have to be transparent but it is more user friendly if it is transparent.

The device is preferably provided with a removable plug for sealing the aperture in the splash guard.

The removable plug is preferably made of rubber or of glass or solid transparent plastic, for example the removable plug may be made of the same material as the splash guard. The plug prevents liquid splashing out during the chilling phase, i.e. when the device is not being used by a subject.

With a plug in place on the splash guard covering the aperture, the splash guard should not be fully airtight as the liquid needs to find a return path to the liquid carrier vessel below in order to avoid creating a vacuum and reducing the flow rate of the liquid stream. In order to prevent air locks, the splash guard does not fully cover the entire opening of the liquid carrier vessel. In a preferred embodiment, one or more edges of the splash guard are curved inwards to create one or more gaps between the perimeter of the splash guard and the inside wall of the liquid carrier vessel. Alternatively or additionally, the splash guard could be slightly smaller than the liquid carrier vessel opening and, for example, be supported by legs which rest on the base of the liquid carrier vessel or rest on protrusions extending into the liquid carrier vessel close to the top thereof. Alternatively or additionally, the splash guard could have a plurality of small holes around the edge or near to its edge to ensure an air lock does not ensue.

The chiller means for cooling the liquid and for maintaining the liquid at a predetermined temperature is configured for connection to a power source or has its own power unit. The chiller means is arranged to cool, or warm, liquid within the liquid carrier vessel to the predetermined temperature and maintain the liquid within a few degrees of the predetermined temperature, e.g. within 1° C.

In a preferred embodiment, the predetermined temperature is in the range of from about 5° C. to about 40° C., particularly preferably from about 10° C. to about 25° C., e.g. 16° C.

In a preferred embodiment, the chiller means is an electrical, e.g. battery powered, chiller, for example comprising a gas filled compressor, a heat exchanger and a fan cooled radiator. Alternatively, the chiller means is a Peltier device. Selection and incorporation of such chiller means will be apparent to those skilled in the art.

The jet works via the Venturi principle. There is a section inside the jet which narrows thereby increasing the flow rate and causing air to be sucked in through the air valve. If the air valve is closed, liquid alone flows through the jet. The whirling movement of the liquid creates a massage effect on the skin surface. As the air valve is opened, more and more air is sucked in and the mixing of air with liquid increases the intensity of the massage effect. Air and liquid flowing through the jet produces more vigorous massage compared to liquid alone. The more turbulence produced, the more the cooling by convection is increased, akin to a ‘wind chill’ effect, resulting in a more penetrating cold.

Preferably, the device has one air valve per jet such that the flow rate of each jet can be adjusted individually.

The pump is a non-submersible conventional structure powered by a conventional electric motor, optionally located within the pump. The pump is a circulating pump operatively connected to a power source for circulation and movement of the liquid through the jet. Selection and incorporation of such a pump will be apparent to those skilled in the art.

The conduit means are each independently preferably flexible hoses. However, this is not to be considered limiting and rigid or flexible pipework may be used. Flexible hoses are preferred over rigid pipework as this minimises the need for elbows and can prevent issues caused by vibration, e.g. cracking of the pipes or pipe fittings.

The aperture in the splash guard is of a size to allow direct contact of a subject's exposed glabrous skin surface with the jet stream.

The aperture is located above the jet such that the aperture, preferably the centre of the aperture, and the vessel inlet are aligned.

In a preferred embodiment, the aperture in the splash guard is circular with a diameter in the range of from about 6 cm to about 9 cm, i.e. about the size of the palm of an adult hand. In a particularly preferred embodiment, the aperture in the splash guard is circular with a diameter of about 7 cm. However, this is not considered to be limiting and the aperture may be of any size suitable to allow a subject to contact a glabrous skin surface with chilled liquid in the liquid carrier vessel. To accommodate individuals with smaller hands an insert may be used to reduce the diameter of the aperture.

In another preferred embodiment, the device further comprises a flexible gasket for placement on the top surface of the splash guard such that it surrounds the aperture. The flexible gasket may be detachable or may be permanently attached to the splash guard. When a subject place their exposed glabrous skin surface, e.g. the palm of one of their hands, over the aperture, the flexible gasket moulds to the contour of the hand to minimise the amount of liquid escaping through the aperture. The flexible gasket is preferably made of soft sponge-like material such as closed cell neoprene. The flexible gasket has a shape corresponding to that of the aperture, for example in the embodiment wherein the aperture is circular, the flexible gasket is preferably of circular cross-section, e.g. a flange gasket.

When using the device to cool the palm of a subject's hand, the hand may be completely uncovered or may be gloved with the palm exposed. In the latter embodiment, the glove assists in maintaining the temperature of the rest of the hand and to aid in the reduction of significant vasoconstriction in subcutaneous blood vessels, thereby maintaining the blood flow to the hand to facilitate maximum heat extraction.

Similarly, when the subject's glabrous skin surface is the sole of the foot, the rest of the foot may be socked with the sole exposed.

When using the device to cool the palm of a completely uncovered hand or foot, the device preferably further comprises means for warming the rest of the hand, for example means for directing heated air to the upper hand surface, i.e. to the back of the hand.

In a preferred embodiment, the device comprises a plurality of inlets in the liquid carrier vessel base, each inlet housing a jet, and the splash guard comprises a corresponding plurality of apertures, wherein each aperture independently is located above a jet and in line with the inlet housing the jet. For example, the device may comprise two jets and a splash guard with two apertures. This will allow two people to use the device simultaneously as only one glabrous surface, e.g. palm, of each person needs to be cooled to achieve the desired core body temperature manipulation, e.g. reduction.

In a preferred embodiment, the device is compact and fully self-contained example with the liquid carrier vessel being isolated from the remaining components. However this is not to be considered limiting and the device may alternatively be modular, for example with the liquid carrier vessel being in a separate unit from the pump and chiller means.

If desired, the device can be permanently installed with plumbing connections for filling the liquid carrier vessel and draining liquid therefrom or it may be a mobile unit independent of plumbing connections. The device is preferably portable, particularly preferably wherein the device is provided with wheels.

The external dimensions of the device may be in the range of from about 60 to about 80 cm long, about 35 to about 45 cm wide and about 60 to about 80 cm high. However, this is not to be considered limiting and the volume capacity may be larger or smaller depending on the intended number of simultaneous users.

The device preferably comprises actuators such as control switches or knobs configured to turn the pump and chiller means on and off independently and to adjust the predetermined temperature, preferably switches suitable for wet environments.

In a preferred embodiment, the device further comprises an electrical controller for example a solid-state controller or a digital controller, e.g. a programmable logic controller, wherein the electrical controller is operatively connected to each of the chiller means and the pump. The electrical controller controls operation of the device by controlling the operation of the pump and the chiller means to create the jet stream at the predetermined temperature and at a predetermined flow rate such that the jet stream reaches the aperture of the splash guard or extends slightly above it, for example 2 or 3 cm above the splash guard.

In a preferred embodiment, the device is provided with a user interface in electronic communication with the chiller means for displaying the temperature of the liquid within the liquid carrier vessel or the predetermined temperature or both. The user interface preferably allows the user to amend the predetermined temperature when required. The user interface preferably comprises a digital display.

The liquid for use with the device is preferably water or brine. In the broadest sense, the liquid may be any composition that is not harmful to skin and is capable of attaining a temperature that, upon contact with a subject's glabrous skin surface, results in transfer of energy from the subject to the liquid or from the liquid to the subject.

In a preferred embodiment, the liquid vessel outlet comprises a drain pipe for discharging liquid from the vessel after use, particularly preferably a drain pipe with a manual valve.

The device is configured both to ensure that the top of the jet stream does not reach the bottom of the splash guard when the air valve is closed and that the top of the jet stream reaches above the splash guard (if not prevented, e.g. by having a hand in place over the aperture or by closing the aperture with a plug) when the air valve is open. This is achieved by controlling the flow rate of the liquid, i.e. by selecting the size of the pump, the diameter of the conduit means and the dimensions of the jet. Such selection will be apparent to those skilled in the art.

In another aspect, the invention provides a method for manipulating core body temperature, for example for recovery during breaks in playing sport, by using the device described herein.

The method preferably comprises the following steps:
(a) filling the liquid carrier vessel, preferably to a predetermined minimum liquid level indicator, with liquid either through the aperture in the splash guard or through the top opening of the liquid carrier vessel if the splash guard is not present;
(b) if the splash guard is not present, placing the splash guard over the top opening;
(c) inserting the removable plug into the splash guard aperture;
(d) checking the air valve is fully closed;
(e) turning on the pump to circulate the liquid wherein the flow rate of the liquid discharged by the jet is such that the liquid does not reach the splash guard;
(f) removing the plug from the splash guard to expose the aperture therein;
(g) resting a subject's extremity on the splash guard such that an exposed glabrous skin surface, e.g. palm of hand, is located over the aperture in the splash guard;
(h) opening the air valve to increase the flow rate of the liquid being discharged from the or each jet such that the liquid reaches the subject's exposed glabrous skin surface.

At the end of a session, the air valve should be closed before the subject removes their skin surface, e.g. palm of hand. The above steps may then be repeated for next subject or, if use of the device is no longer required, the plug can be replaced and the power switched off.

In a preferred embodiment, the method further comprises the step, after removal of the plug and prior to location of the subject's exposed glabrous skin surface, of placing a flexible gasket onto the top surface of the splash guard such that the gasket surrounds the aperture. Naturally, if the splash guard already has a permanently attached gasket, this step is superfluous. The gasket minimises the amount of liquid escaping from the vessel through the aperture during operation of the device.

The method preferably further comprises the following steps to pre-chill or heat the liquid to attain a desired predetermined temperature prior to use:
(a) covering the liquid carrier vessel opening with the splash guard and plug;
(b) activating the pump and the chiller means;
(c) checking the predetermined temperature and adjusting if necessary;
(d) checking the air valve is fully closed and closing if necessary.

Once the liquid in the vessel has reached the desired predetermined temperature, the device is ready for use. The above liquid temperature setting steps preferably take around 5 minutes but could be longer or shorter depending on both the predetermined and ambient temperatures.

Preferably, the method further includes the step of displaying the temperature of the liquid. This requires the device to have a display as described above.

To maximise the effect produced by the device, the cold stimulus provided by the jet stream is preferably controlled to avoid triggering the anti-heat loss vasoconstriction response. This is achieved by only exposing glabrous skin to the cold stimulus and keeping the rest of the subject's skin dry in order to maintain normal skin temperature. By “normal skin temperature” is meant a temperature in the range of from about 28° C. to about 32° C. as this can vary according to ambient temperature and other external factors.

Ideally, the temperature of the skin not intended to be exposed to the cold stimulus should not reduce by more than 2° C., preferably by not more than 1° C., in order to maintain blood flow and avoid the triggering of vasoconstriction, which would reduce the cooling effect.

In a preferred embodiment, the glabrous surface is the palm of the hand and the rest of the hand is kept warm and dry, for example by protection with an insulated glove, preferably wherein the glove is made of neoprene or similar material. This avoids vasoconstriction and does not restrict the flow of the subject's blood to the palm of their hand.

In an alternative embodiment, the rest of the hand is kept warm and dry by heating with warm air, for example from an external heat source such as a hairdryer, or by recirculating warm air produced by the chiller means.

Wearing a glove as detailed above can be beneficial in maintaining skin temperature particularly if the subject has poor circulation or if their hands tend to be more sensitive to the cold. There may be a gender variation as women typically have lower blood pressure and lower hand temperatures than men. There may also be ethnic variations. However, the device allows for rapid temperature modification to suit individual requirements.

### BRIEF DESCRIPTION OF THE DRAWINGS

Certain preferred embodiments of the present invention will now be described, by way of example only, with reference to the accompanying drawings in which:

FIG. 1 shows a perspective view of a preferred embodiment of the device according to the invention;

FIG. 2 shows a schematic view of the device shown in FIG. 1;

FIG. 3 shows a plan view of another preferred device according to the invention;

FIG. 4 shows a cross-sectional view of the device shown in FIG. 1;

FIG. 5 shows the device shown in FIG. 1 in use;

FIG. 6a shows a plan view of a preferred plug for closing the splash guard aperture;

FIG. 6b shows a cross-sectional view of the plug in FIG. 6a in place closing a splash guard aperture; and

FIG. 7 shows a view from below of a preferred glove for use with the device.

### DETAILED DESCRIPTION

Various embodiments of the present invention will be described in detail with reference to the drawings, where like reference numeral represent like parts and assemblies throughout the several views.

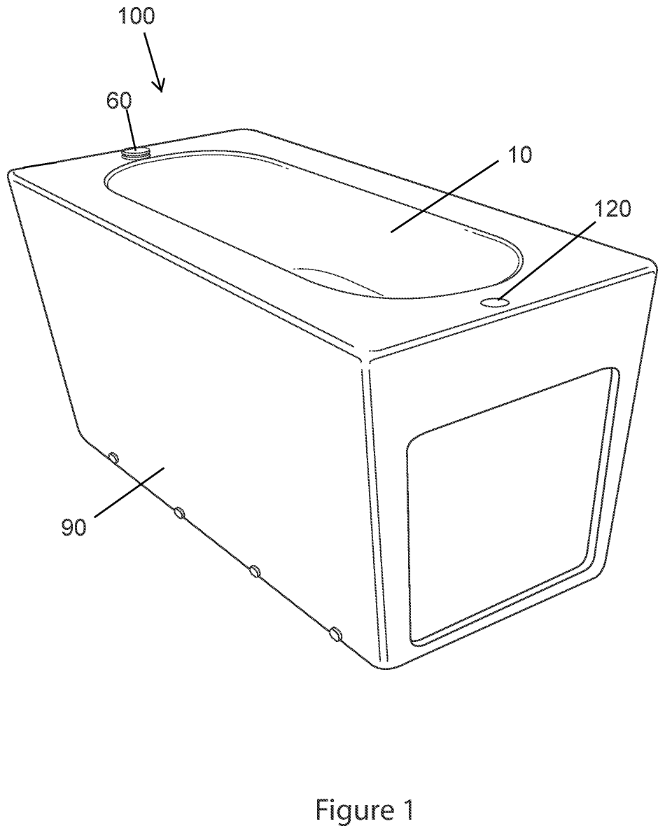

Referring to the drawings, FIGS. 1, 2, 4 and 5 show a preferred device according to the invention, generally referred to herein by reference numeral 100, for maintaining core body temperature of a subject by both convection and conduction. This is achieved by contact of an exposed glabrous skin surface belonging to the subject with a cold stimulus provided by jet stream 110.

As shown in FIG. 1, device 100 comprises main housing 90 with open topped liquid carrier vessel 10 for retaining a liquid L. Power switch 120 for turning device 100 on and off and air valve 60 are visible at the top of housing 90 adjacent the opening of liquid carrier vessel 10.

Liquid carrier vessel 10 is in the form of a shell with a well, for example like a small bathtub or basin, e.g. an elongated trough, Liquid carrier vessel 10 is open at the top and has inlet 12 and outlet 13 in base 102 thereof.

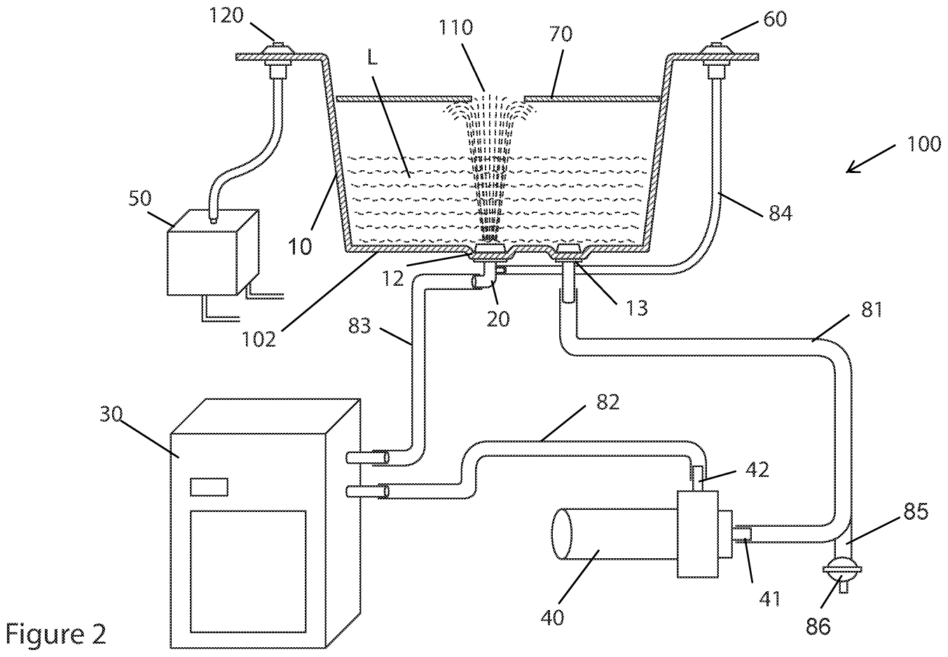

As shown schematically in FIG. 2, device 100 further comprises jet 20 for discharging liquid and air into liquid carrier vessel 10, chiller 30 for cooling liquid L and maintaining it at the predetermined temperature, pump 40 for circulating the liquid, electrical controller 50 for powering chiller means 30 and pump 40, air valve 60 for controlling the amount of air flowing through jet 20 and removable splash guard 70 with aperture 71.

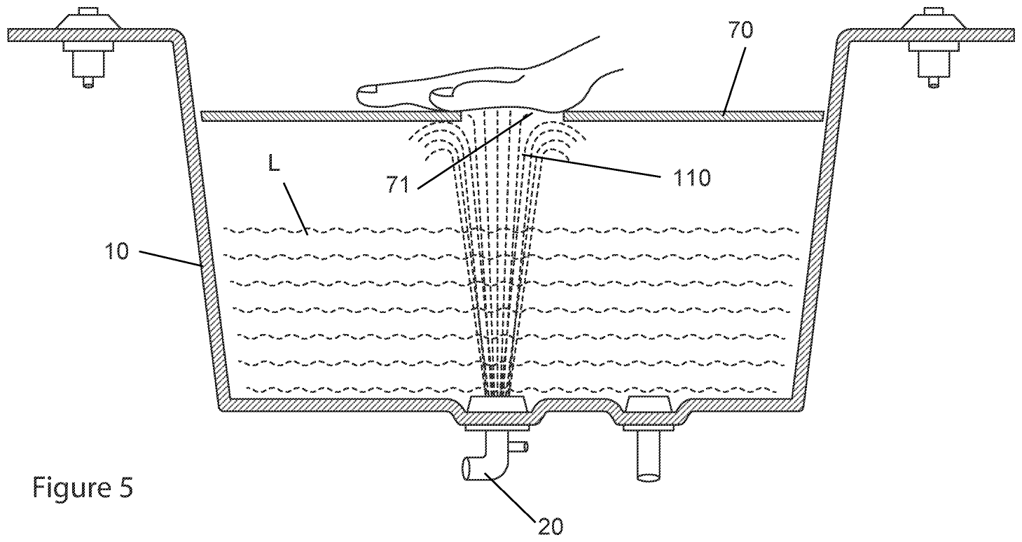

As shown in FIG. 5, aperture 71 is of a size to allow direct contact of a subject's exposed glabrous skin surface with jet stream 110.

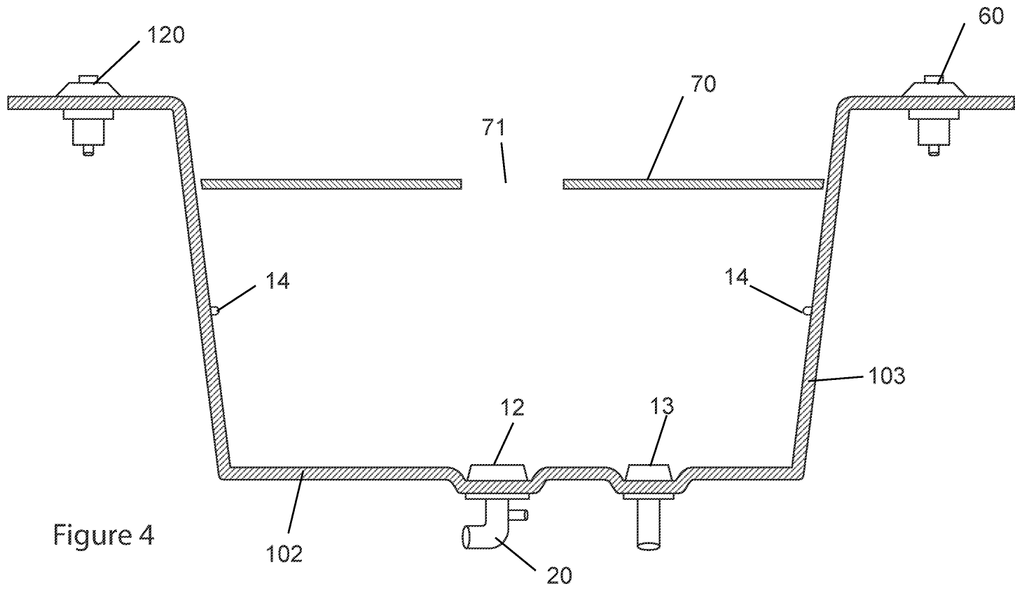

Side wall 103 of liquid carrier vessel 10 has minimum liquid level indicator 14, visible in FIG. 4, to guide a user as to the minimum amount of liquid L required to operate device 100. In the preferred embodiment shown in the Figures, minimum liquid level indicator 14 is a raised area on sidewall 103.

In order to avoid creating a vacuum or reducing the flow rate of jet stream 110 during use of device 100, splash guard 70 does not cover the entire opening of liquid carrier vessel L. In the preferred embodiment shown in the Figures, this is achieved due by the edges of the splash guard being curved inwards so that it is only the corners, rather than the entire outer edge, of splash guard 70 that contact the inside of liquid carrier vessel 10.

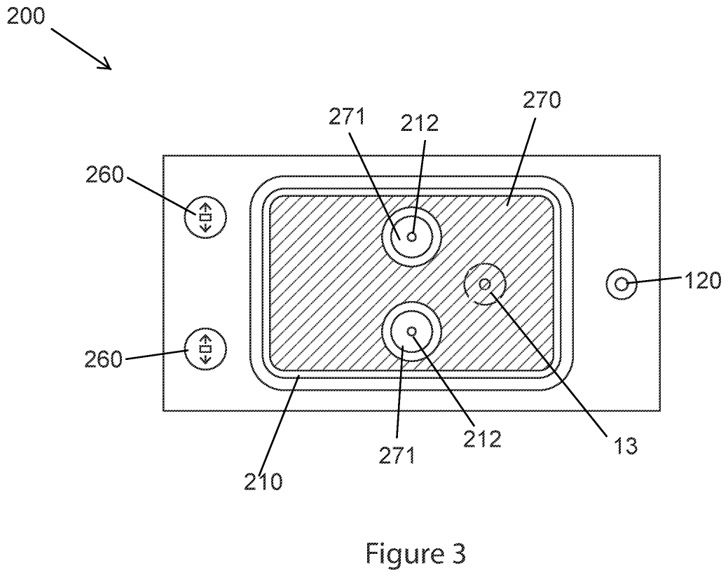

A preferred alternative embodiment is shown in FIG. 3. Device 200 differs from device 100 in that splash guard 270 of device 200 has two apertures 271 and two jets 220. Liquid vessel carrier 210 therefore has two inlets 212 and device 200 has two air valves 260 such that the flow rate of each jet can be adjusted individually. As shown in FIG. 3, splash guard 270 is transparent. Splash guard 70 in the remaining Figures is also transparent.

Each jet 20, 220 independently works via the Venturi principle. Inside each jet 20, 220 independently there is a section which narrows thereby increasing the flow rate of liquid passing therethrough and causing air to be sucked in through the corresponding air valve 60, 260.

Chiller 30 is arranged to cool, or warm, liquid L within liquid carrier vessel 10 to the predetermined temperature and maintain the liquid within a few degrees, e.g. within 1° C., of the predetermined temperature.

Pump 40 is a non-submersible conventional circulating pump configured to suck liquid into suction side 41 from outlet 13 and propel liquid out of pressure side 42 through jet 20 via chiller 30.

As shown in FIG. 2, pipe 81 interconnects outlet 13 with suction side 41 of pump 40, pipe 82 interconnects chiller 30 with pressure side 42 of pump 40, pipe 83 interconnects chiller 30 with jet 20 and pipe 84 interconnects jet 20 with air valve 60. Pipe 81 is further attached to drain pipe 85 with manual valve 86 for discharging liquid L from vessel 10 after use of device 100.

Each of pipes 81, 82, 83 and 84 are preferably ¾ inch (1.9 cm) diameter flexible hoses. However, this is not to be considered limiting and rigid or flexible pipework may be used.

Electrical controller 50 provides power to chiller 30 and pump 40 to thus create jet stream 110 at the predetermined temperature and at a predetermined flow rate.

Electrical controller 50 may be a solid-state controller or a digital controller such as a programmable logic controller and is operatively connected to each of chiller 30 and pump 40. Electrical controller 50 controls operation of device 100 by controlling the temperature of liquid L in vessel 10 and the operation of pump 40 thus creating jet stream 110 at a predetermined temperature and predetermined flow rate.

In the preferred embodiment shown in FIG. 1, jet 20, chiller 30, pump 40, electrical controller 50 and pipes 81, 82, 83 and 84 are located within housing 90.

Wheels (not shown) are preferably located in or at the base of housing 90 to increase portability of device 100.

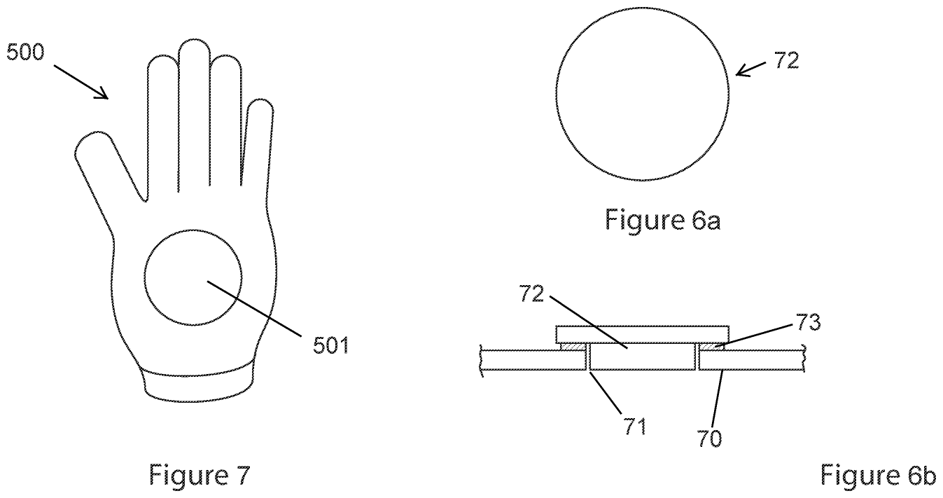

As shown in FIG. 6b, removable plug 72 is used to seal aperture 71 when device 100 is not in use and flexible gasket 73 is placed on, or permanently attached to, the top surface of splash guard 70 such that it surrounds aperture 71. Plug 72 prevents liquid L splashing out during the chilling phase, i.e. when device 100 is not being used by a subject. Device 200 requires two removable plugs (not shown).

In order to use device 100 or 200, liquid carrier vessel 10, 210 is filled to predetermined minimum fill line 14 with liquid L either through one of apertures 71, 271 or through its top opening if splash guard 70, 270 is not yet present.

In order to pre-chill or heat liquid L to attain the desired temperature prior to use, once vessel 10, 210 is filled, it is covered with splash guard 70 and plugs 72 are inserted into each aperture 71, 271 independently. Electrical controller 50 is then switched on via power switch 120 to activate pump 40 and chiller 30. The predetermined temperature of liquid L in vessel 10, 210 is then checked and adjusted if necessary, using an actuator (not shown). A suitable actuator may for example be in the form of up/down buttons or a dial. Once the predetermined temperature has been reached, device 100, 200 is ready for use.

Ensuring the air valves are closed, pump 40 is then turned on to circulate the liquid wherein the flow rate of the liquid discharged by jets 20 is such that jet streams 110 do not reach the bottom of splash guard 70, 270. Plugs 72 are then removed from splash guard 70, 270 to expose apertures 71, 271. A subject then places the exposed palm of one hand over one aperture 71, 271 and the corresponding air valve 60, 260 is opened to increase the flow rate of the liquid being discharged from jet 20, 220 such that jet stream 110 reaches the palm of the subject's hand.

At the end of a session, air valve 60, 260 should be closed before the subject removes their hand. The above steps may then be repeated for next subject or, if use of device 100 is no longer required, plug 72 can be replaced and electrical controller 50 switched off.

When using device 100, 200 the palm of the subject's hand is the only portion of the hand that is exposed and the rest of the hand is kept warm and dry, for example by protection with insulated glove 500 with hole 501 as shown in FIG. 7. This avoids vasoconstriction and does not restrict the flow of the subject's blood to the palm of their hand.

It is to be understood that the invention is not limited to the specific details described herein which are given by way of example only and that various modifications and alterations are possible without departing from the scope of the invention as defined in the appended claims.

1-13. (canceled)

14. An apparatus for manipulating core body temperature of a human, the apparatus comprising:
a liquid carrier vessel comprising an opening and adapted to receive a liquid, the liquid carrier vessel having an inlet and an outlet;
a splash guard for covering the majority of the opening of the liquid carrier vessel and for resting thereon a human extremity having an exposed glabrous skin surface, wherein the splash guard is removable;
a chiller for cooling the liquid received in the liquid carrier vessel and for maintaining the liquid at a predetermined temperature;
a jet in the vessel inlet for discharging the liquid, or a combination of the liquid and air into the liquid carrier vessel;
an air valve for controlling the amount of air flowing through the jet;
a pump configured to suck the liquid from the liquid carrier vessel via the vessel outlet into a suction side of the pump and propel the liquid out of a pressure side of the pump into the liquid carrier vessel through the vessel inlet via the chiller to create a jet stream within the liquid carrier vessel at the predetermined temperature, wherein the splash guard comprises an aperture to allow the jet stream to contact the exposed glabrous skin surface during operation of the apparatus, and wherein the aperture of the splash guard is located directly above the jet;
a first conduit interconnecting the vessel outlet with the suction side of the pump;
a second conduit interconnecting the chiller with the pressure side of the pump;
a third conduit interconnecting the chiller with the jet; and
a fourth conduit interconnecting the jet with the air valve.

15. The apparatus of claim 14, wherein the apparatus comprises a plurality of jets and the splash guard comprises a corresponding plurality of apertures.

16. The apparatus of claim 15, wherein the apparatus comprises one air valve per jet.

17. The apparatus of claim 15, wherein the apparatus further comprises a plurality of removable plugs for sealing the apertures of the splash guard.

18. The apparatus of claim 14, wherein the apparatus comprises two jets and two air valves and the splash guard comprises two apertures.

19. The apparatus of claim 14, wherein the apparatus further comprises one or more removable plugs for sealing the aperture of the splash guard.

20. The apparatus of claim 14, wherein the splash guard is smaller than the opening of the liquid carrier vessel and is supported on legs.

21. The apparatus of claim 14, wherein the splash guard has sides which are curved inwardly thereby forming gaps that are located between the splash guard and the vessel.

22. The apparatus of claim 14, wherein the splash guard comprises a removable plug.

23. The apparatus of claim 14, wherein the splash guard comprises a flexible gasket.

24. A method for manipulating core body temperature of a human using an apparatus, the apparatus comprising:
a liquid carrier vessel comprising an opening and adapted to receive a liquid, the liquid carrier vessel having an inlet and an outlet;
a splash guard for covering the majority of the opening of the liquid carrier vessel and for resting thereon a human extremity having an exposed glabrous skin surface, wherein the splash guard is removable;
a chiller for cooling the liquid received in the vessel and for maintaining the liquid at a predetermined temperature;
a jet in the vessel inlet for discharging the liquid, or a combination of the liquid and air into the liquid carrier vessel;
an air valve for controlling the amount of air flowing through the jet; and
a pump configured to suck the liquid from the liquid carrier vessel via the vessel outlet into a suction side of the pump and propel the liquid out of a pressure side of the pump into the liquid carrier vessel through the vessel inlet via the chiller to create a jet stream within the liquid carrier vessel at the predetermined temperature, wherein the splash guard comprises an aperture to allow the jet stream to contact the exposed glabrous skin surface during operation of the apparatus, and wherein the aperture of the splash guard is located directly above the jet;
the method comprising:
placing the splash guard over the opening of the liquid carrier vessel before or after the liquid carrier vessel is filled with the liquid;
inserting the removable plug into the aperture of the splash guard;
checking the air valve is fully closed;
turning on the pump to circulate the liquid wherein the flow rate of the liquid discharged by the jet is such that the liquid does not reach the splash guard;
removing the plug from the splash guard to expose the aperture of the splash guard;
resting the human's extremity on the splash guard such that an exposed glabrous skin surface is located over the aperture of the splash guard; and
opening the air valve to increase the flow rate of the liquid being discharged from the jet such that the liquid reaches the exposed glabrous skin surface.

25. The method of claim 24, the method further comprising, after removing the plug, placing a flexible gasket onto the top surface of the splash guard such that the gasket surrounds the aperture of the splash guard.

26. The method of claim 24, the method further comprising pre-chilling or heating the liquid to attain the predetermined temperature prior to use by:
covering the opening of the liquid carrier vessel with the splash guard and plug;
activating the pump and the chiller;
checking the predetermined temperature and adjusting if necessary; and
checking the air valve is fully closed and closing if necessary.

27. The method of claim 24, the method further comprising filling the liquid carrier vessel with liquid either through the aperture of the splash guard or through the opening of the liquid carrier vessel.

28. The method of claim 24, wherein the apparatus further comprises:
a first conduit interconnecting the vessel outlet with the suction side of the pump;
a second conduit interconnecting the chiller with the pressure side of the pump;
a third conduit interconnecting the chiller with the jet; and
a fourth conduit interconnecting the jet with the air valve.

29. A system for manipulating core body temperature of a human, the system comprising:
a device comprising:
a liquid carrier vessel comprising an opening and adapted to receive a liquid, the liquid carrier vessel having an inlet and an outlet;
a splash guard for covering the majority of the opening of the liquid carrier vessel and for resting thereon a human extremity having an exposed glabrous skin surface, wherein the splash guard is removable;
a chiller for cooling the liquid received in the vessel and for maintaining the liquid at a predetermined temperature;
a jet in the vessel inlet for discharging the liquid, or a combination of the liquid and air into the liquid carrier vessel;
an air valve for controlling the amount of air flowing through the jet; and
a pump configured to suck the liquid from the liquid carrier vessel via the vessel outlet into a suction side of the pump and propel the liquid out of a pressure side of the pump into the liquid carrier vessel through the vessel inlet via the chiller to create a jet stream within the liquid carrier vessel at the predetermined temperature, wherein the splash guard comprises an aperture to allow the jet stream to contact the exposed glabrous skin surface during operation of the apparatus, and wherein the aperture of the splash guard is located directly above the jet; and
a glove comprising a hole configured to expose the glabrous skin surface of a human hand.

30. The system of claim 29, wherein the device further comprises:
a first conduit interconnecting the vessel outlet with the suction side of the pump;
a second conduit interconnecting the chiller with the pressure side of the pump;
a third conduit interconnecting the chiller with the jet; and
a fourth conduit interconnecting the jet with the air valve.
